(19) United States
(12) Patent Application Publication
(10) Pub. No.: US 2016/0058686 A1
(43) Pub. Date: Mar. 3, 2016

(54) COSMETIC HAIRCARE COMPOSITION FOR RELAXING CURLS AND/OR FOR REDUCING VOLUME

(71) Applicant: L'OREAL, Paris (FR)

(72) Inventors: Myriam MELLUL, Paris (FR); Laurence PAUL, Saint Leu la Forêt (FR); Aude LIVOREIL-DJAMPOU, Le Bourget (FR)

(21) Appl. No.: 14/785,102

(22) PCT Filed: Apr. 15, 2014
(86) PCT No.: PCT/EP2014/057647

§ 371 Date: Oct. 16, 2015

(30) Foreign Application Priority Data
Apr. 19, 2013 (FR) 1353593
Apr. 19, 2013 (FR) 1353595

(51) Int. Cl. A61K 8/891; A61K 8/41; A61K 8/73; A61K 8/34; A61K 8/67; A61K 8/43; A61K 8/36; A61Q 5/04; A61K 8/19; A61K 8/22; A61K 8/42; A61K 8/46

(52) U.S. Cl. CPC A61K 8/891; A61K 8/42; A61K 8/416; A61K 8/732; A61K 8/342; A61K 8/46; A61K 8/43; A61K 8/41; A61K 8/36; A61Q 5/04; A61K 8/19; A61K 8/22; A61K 8/676

(57) ABSTRACT

The present invention relates to a cosmetic composition comprising: a) one or more amphoteric or zwitterionic surfactants, b) one or more cationic surfactants, c) one or more sulfureous reducing agents, and d) one or more alkaline agents, the weight ratio between the total amount of the amphoteric or zwitterionic surfactant(s), on the one hand, and the total amount of the cationic surfactant(s), on the other hand, being greater than or equal to 1.2. The present invention also relates to processes using this composition. The invention also relates to the use of the composition according to the invention for relaxing the curls and/or reducing the volume of keratin fibres, preferably the hair.

The present invention relates to a composition for the cosmetic treatment of keratin fibres, which is intended in particular for relaxing the curls and/or reducing the volume of the said fibres and which comprises at least one amphoteric or zwitterionic surfactant, at least one cationic surfactant, at least one sulfureous reducing agent and at least one alkaline agent.

The present invention also relates to a cosmetic process for treating keratin fibres such as the hair, using the said composition.

According to a particular embodiment, this process consists in relaxing the curls of and/or in straightening keratin fibres, and comprises a step of applying to the keratin fibres the composition according to the invention, followed by a step of applying to the keratin fibres a second composition comprising one or more oxidizing agents and/or one or more reductones.

The invention also relates to the use of this composition for relaxing the curls and/or reducing the volume of keratin fibres.

Finally, a subject of the invention is a multi-compartment device or “kit” that is suitable for performing the process according to the invention.

To obtain permanent reshaping of the hair such as straightening of the hair, uncurling or relaxing of the curls, the technique most commonly used consists, in a first stage, in opening the —S—S— disulfide bonds of keratin (keratocystine) generally by means of a basic composition containing a sulfureous reducing agent (reduction step), and then, after having rinsed the head of hair thus treated, generally with water, in reconstituting, in a second stage, the said disulfide bonds by applying to the hair, which has been placed under tension beforehand, an oxidizing composition (oxidation step, also known as the fixing step) so as finally to give the hair the desired shape.

The new shape given to the hair by such a chemical treatment is eminently long-lasting and especially withstands washing with water or shampoos, as opposed to the simple standard techniques of temporary reshaping, such as hairsetting.

Many products intended for straightening or uncurling the hair or for relaxing curls exist on the market.

Products intended for straightening or uncurling the hair are generally formulated with thiols and alkaline agents, whereas products intended for relaxing curls tend rather to contain products such as cysteine and derivatives thereof.

These products are generally applied to curly or voluminous hair in order to obtain more or less pronounced straightening and a reduction in the volume and mass of the head of hair.

The application of these products is generally long, with a longer or shorter leave-on time depending on the product, the type of hair and the desired effect. It requires precise know-how.

Furthermore, reducing agents are generally used in high concentrations, which may lead to more or less pronounced degradation of the hair fibre, in particular when these fibres are coloured.

In addition, the use of such a straightening treatment is a choice that is generally drastic for the consumer, who cannot regulate the level of straightening at will.

Finally, the use of such a straightening treatment generally gives rise to odour problems that may prove to be troublesome to the consumer.

There is thus a real need to develop hair straightening treatments, which are lighter than those existing in the prior art, which not only degrade the hair fibre less, but also are easy to apply, and allow modulation of the straightening effect, for example by making use of repeated applications.

The Applicant has now discovered that the introduction of certain sulfureous active agents into a particular care base makes it possible to satisfy the abovementioned objectives.

In particular, it has discovered that by introducing at least one sulfureous reducing agent and at least one alkaline agent into a care base containing a particular combination of surfactants based on an amphoteric or zwitterionic surfactant, and a cationic surfactant, while respecting a particular weight concentration ratio between these two types of surfactants, a care composition having straightening activity on the hair may be obtained.

One subject of the present invention is thus a cosmetic composition comprising:
a) one or more amphoteric or zwitterionic surfactants,
b) one or more cationic surfactants,
c) one or more sulfureous reducing agents, and
d) one or more alkaline agents,

the weight ratio between the total amount of the amphoteric or zwitterionic surfactant(s), on the one hand, and the total amount of the cationic surfactant(s), on the other hand, being greater than or equal to 1.2.

The application of such a composition to keratin fibres is rapid and simple, and does not require any particular know-how.

Furthermore, the application of this composition makes it possible to obtain a relaxation of curls and/or a reduction of the volume of the head of hair that is gradual. In particular, it is possible to modulate the desired effect, by successive applications of such a composition.

In particular, the composition according to the invention may be used as a straightening maintenance product, especially between two straightening operations.

In addition, the composition according to the invention has good detergency properties and very good working qualities.

Finally, the treatment by means of the composition according to the invention virtually does not degrade hair fibres.

The invention also relates to a cosmetic process for treating keratin fibres, comprising the application to the said wet or dry fibres, in one or more stages, of the composition according to the invention.

According to a particular embodiment, the invention relates to a process for relaxing the curls of and/or for straightening keratin fibres, such as the hair, and which comprises:

a) a step of applying to the keratin fibres the composition according to the invention; followed by

b) a step of applying to the keratin fibres a second composition (B) comprising one or more oxidizing agents and/or one or more reductones.

The implementation of such a process is rapid and simple, and does not require any particular know-how.

Furthermore, the implementation of this process makes it possible to obtain a relaxation of curls and/or a reduction of the volume of the head of hair that is gradual. In particular, it is possible to modulate the desired effect, by successive implementations of such a process.

In particular, this process may be performed in order to maintain the straightening, especially between two straightening operations.

Also, the implementation of the process according to the invention does not degrade hair fibres.

Finally, the process according to the invention makes it possible to substantially reduce the unpleasant odours derived from performing a standard curl relaxation and/or straightening process.

Other characteristics and advantages of the invention will emerge more clearly on reading the description and the examples that follow.

The composition according to the invention advantageously comprises water or a mixture of water and of one or more cosmetically acceptable solvents chosen from C1-C4 lower alcohols, such as ethanol, isopropanol, tert-butanol or n-butanol, polyols such as propylene glycol, polyol ethers, C5-C10 alkanes, C3-C4 ketones, such as acetone and methyl ethyl ketone, C1-C4 alkyl acetates, such as methyl acetate, ethyl acetate and butyl acetate, dimethoxyethane and diethoxyethane, and mixtures thereof.

The amphoteric or zwitterionic surfactant(s) that may be used in the composition according to the invention may especially be derivatives of aliphatic secondary or tertiary amines, optionally quaternized, in which derivatives the aliphatic group is a linear or branched chain comprising from 8 to 22 carbon atoms, the said amine derivatives containing at least one anionic group, such as a carboxylate, sulfonate, sulfate, phosphate or phosphonate group. Mention may be made in particular of (C₈-C₂₀)alkylbetaines such as cocoylbetaine, sulfobetaines, (C₈-C₂₀)alkylamido(C₂-C₈)alkylbetaines such as cocoylamidopropylbetaine or (C₈-C₂₀)alkylamido(C₆-C₈)-alkylsulfobetaines, and mixtures thereof.

Among the derivatives of aliphatic secondary or tertiary amines, optionally quaternized, that may be used, as defined above, mention may also be made of the compounds of respective structures (I), (II) and (IIa) below:

Ra-C(O)—NHCH₂CH₂—N⁺(Rb)(Rc)-CH₂COO⁻,M⁺,X⁻ (I)

in which formula (I):

Ra represents a C₁₀-C₃₀ alkyl or alkenyl group derived from an acid Ra-COOH preferably present in hydrolysed coconut oil, or a heptyl, nonyl or undecyl group;

Rb represents a beta-hydroxyethyl group; and

Rc represents a carboxymethyl group;

M⁺ represents a cationic counterion derived from an alkali metal or alkaline-earth metal, such as sodium, an ammonium ion or an ion derived from an organic amine; and

X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylarylsulfonates, in particular methyl sulfate and ethyl sulfate; or alternatively M⁺and X⁻ are absent;

Ra′-C(O)—NHCH₂CH₂—N(B)(B′) (II)

in which formula (II):

B represents the group —CH₂—CH₂—O—X′;

B′ represents the group —(CH₂)_{z}Y′, with z=1 or 2;

X′ represents the group —CH₂—COOH, CH₂—COOZ′, —CH₂CH₂—COOH or —CH₂CH₂—COOZ′, or a hydrogen atom;

Y′ represents the group —COOH, —COOZ′, CH₂CH(OH)SO₃H or the group —CH₂CH(OH)SO₃Z′;

Z′ represents a cationic counterion derived from an alkali metal or alkaline-earth metal, such as sodium, an ammonium ion or an ion derived from an organic amine;

Ra′ represents a C₁₀-C₃₀ alkyl or alkenyl group of an acid Ra′-COOH, which is preferably present in coconut oil or in hydrolysed linseed oil, or an alkyl group, especially a C₁₇ group and its iso form, or an unsaturated C₁₇ group.

The compounds of this type are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate and the cocoamphodipropionate sold by the company Evonik Goldschmidt under the trade name Rewoteric AM KSF 40.

Ra″-NH—CH(Y″)—(CH₂)ₙ—C(O)—NH—(CH₂)_{n′}—N(Rd)(Re) (IIa)

in which formula (IIa):

Y″ represents the group —COOH, —COOZ″, —CH₂CH(OH)SO₃H or the group —CH₂CH(OH)SO₃Z″;

Rd and Re, independently of each other, represent a C₁-C₄ alkyl or hydroxyalkyl radical;

Z″ represents a cationic counterion derived from an alkali metal or alkaline-earth metal, such as sodium, an ammonium ion or an ion derived from an organic amine;

Ra″ represents a C₁₀-C₃₀ alkyl or alkenyl group of an acid Ra″-COOH which is preferably present in coconut oil or in hydrolysed linseed oil;

n and n′ denote, independently of each other, an integer ranging from 1 to 3;

and mixtures of these compounds.

Among the compounds of formula (IIa), mention may be made of the compound classified in the CTFA dictionary under the name sodium diethylaminopropyl cocoaspartamide and sold by the company Chimex under the name Chimexane HB.

More preferentially, the amphoteric or zwitterionic surfactant(s) are chosen from cocoylbetaine, cocoylamidopropylbetaine and sodium cocoylamidoethyl-N-hydro xyethylaminopropionate.

The above amphoteric or zwitterionic surfactant(s) may be present in an amount ranging from 0.1% to 25% by weight, preferably from 0.5% to 15% by weight and better still from 1% to 10% by weight, relative to the total weight of the composition.

The term “cationic surfactant” means a surfactant that is positively charged when it is contained in the composition according to the invention. This surfactant may bear one or more positive permanent charges or may contain one or more functions that are cationizable in the composition according to the invention.

The cationic surfactant(s) are preferably chosen from optionally polyoxyalkylenated, primary, secondary or tertiary fatty amines, or salts thereof, and quaternary ammonium salts, and mixtures thereof.

The fatty amines generally comprise at least one C₈-C₃₀ hydrocarbon-based chain.

Examples of quaternary ammonium salts that may especially be mentioned include:
those corresponding to the general formula (III) below:

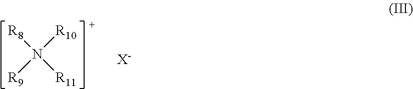

in which the groups R₈ to R₁₁, which may be identical or different, represent a linear or branched, saturated or unsaturated aliphatic group comprising from 1 to 30 carbon atoms, or an aromatic group such as aryl or alkylaryl, at least one of the groups R₈ to R₁₁ denoting a group comprising from 8 to 30 carbon atoms and preferably from 12 to 24 carbon atoms. The aliphatic groups may comprise heteroatoms especially such as oxygen, nitrogen, sulfur and halogens. The aliphatic groups are chosen, for example, from C₁-C₃₀ alkyl, C₂-C₃₀ alkenyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate and C₁-C₃₀ hydroxyalkyl groups; X⁻is an anion chosen from the group of halides, phosphates, acetates, lactates, (C₁-C₄)alkyl sulfates, and (C₁-C₄)alkyl- or (C₁-C₄)alkylarylsulfonates.

Among the quaternary ammonium salts of formula (III), those that are preferred are, on the one hand, tetraalkylammonium salts, for instance dialkyldimethylammonium or alkyltrimethylammonium salts in which the alkyl group contains approximately from 12 to 22 carbon atoms, in particular behenyltrimethylammonium, distearyldimethylammonium, cetyltrimethylammonium or benzyldimethylstearylammonium salts, or, on the other hand, oleocetyldimethylhydroxyethylammonium salts, palmitylamidopropyltrimethylammonium salts, stearamidopropyltrimethylammonium salts and stearamidopropyldimethylcetearylammonium salts. It is particularly preferred to use the chloride salts of these compounds.
quaternary ammonium salts of imidazoline, such as, for example, those of formula (IV) below:

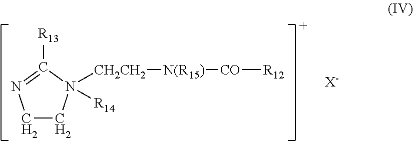

in which R₁₂ represents an alkenyl or alkyl group comprising from 8 to 30 carbon atoms, derived for example from tallow fatty acids, R₁₃ represents a hydrogen atom, a C₁-C₄ alkyl group or an alkyl or alkenyl group comprising from 8 to 30 carbon atoms, R₁₄ represents a C₁-C₄ alkyl group, R₁₅ represents a hydrogen atom or a C₁-C₄ alkyl group, X⁻ is an anion chosen from the group of halides, phosphates, acetates, lactates, alkyl sulfates, alkyl- or alkylaryl-sulfonates in which the alkyl and aryl groups preferably comprise, respectively, from 1 to 20 carbon atoms and from 6 to 30 carbon atoms. R₁₂ and R₁₃ preferably denote a mixture of alkenyl or alkyl groups containing from 12 to 21 carbon atoms, derived for example from tallow fatty acids, R₁₄ preferably denotes a methyl group, and R₁₅ preferably denotes a hydrogen atom. Such a product is sold, for example, under the name Rewoquat® W 75 by the company Rewo;
quaternary diammonium or triammonium salts, in particular of formula (V):

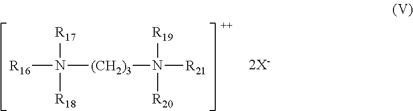

in which R₁₆ denotes an alkyl radical comprising approximately from 16 to 30 carbon atoms, which is optionally hydroxylated and/or interrupted with one or more oxygen atoms, R₁₇ is chosen from hydrogen or an alkyl radical comprising from 1 to 4 carbon atoms or a group (R₁₆ₐ)(R₁₇ₐ)(R₁₈ₐ)N—(CH₂)₃,

R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms, and X⁻ is an anion chosen from the group of halides, acetates, phosphates, nitrates and methyl sulfates. Such compounds are, for example, Finquat CT-P, sold by the company Finetex (Quaternium 89), and Finquat CT, sold by the company Finetex (Quaternium 75),
quaternary ammonium salts containing at least one ester function, such as those of formula (VI) below:

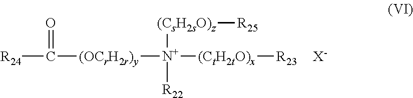

in which:

R₂₂ is chosen from C₁-C₆ alkyl groups and C₁-C₆ hydroxyalkyl or dihydroxyalkyl groups;

R₂₃ is chosen from:
the group

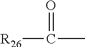

groups R₂₇, which are linear or branched, saturated or unsaturated C₁-C₂₂ hydrocarbon-based groups,
a hydrogen atom,

R₂₅ is chosen from:
the group

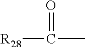

groups R₂₉, which are linear or branched, saturated or unsaturated C₁-C₆ hydrocarbon-based groups,
a hydrogen atom,

R₂₄, R₂₆ and R₂₈, which may be identical or different, are chosen from linear or branched, saturated or unsaturated C₇-C₂₁ hydrocarbon-based groups;

r, s and t, which may be identical or different, are integers ranging from 2 to 6;

y is an integer ranging from 1 to 10;

x and z, which may be identical or different, are integers ranging from 0 to 10;

X⁻ is a simple or complex, organic or mineral anion;

with the proviso that the sum x+y+z is from 1 to 15, that when x is 0 then Rₙ denotes R₂₇, and that when z is 0 then R₂₅ denotes R₂₉.

The alkyl groups R₂₂ may be linear or branched, and more particularly linear.

Preferably, R₂₂ denotes a methyl, ethyl, hydroxyethyl or dihydroxypropyl group, and more particularly a methyl or ethyl group.

Advantageously, the sum x+y+z is from 1 to 10.

When R₂₃ is a hydrocarbon-based group R₂₇, it may be long and contain from 12 to 22 carbon atoms, or may be short and contain from 1 to 3 carbon atoms.

When R₂₅ is an R₂₉ hydrocarbon-based group, it preferably contains 1 to 3 carbon atoms.

Advantageously, R₂₄, R₂₆ and R₂₈, which may be identical or different, are chosen from linear or branched, saturated or unsaturated C₁₁-C₂₁ hydrocarbon-based groups, and more particularly from linear or branched, saturated or unsaturated C₁₁-C₂₁ alkyl and alkenyl groups.

Preferably, x and z, which may be identical or different, have values of 0 or 1.

Advantageously, y is equal to 1.

Preferably, r, s and t, which may be identical or different, are equal to 2 or 3, and even more particularly are equal to 2.

The anion X⁻ is preferably a halide (chloride, bromide or iodide) or an alkyl sulfate, more particularly methyl sulfate. However, use may be made of methanesulfonate, phosphate, nitrate, tosylate, an anion derived from an organic acid, such as acetate or lactate, or any other anion compatible with the ammonium containing an ester function.

The anion X⁻ is even more particularly chloride or methyl sulfate.

Use is made more particularly, in the composition according to the invention, of the ammonium salts of formula (VI) in which:

R₂₂ denotes a methyl or ethyl group,

x and y are equal to 1;

z is equal to 0 or 1;

r, s and t are equal to 2;

R₂₃ is chosen from:
the group

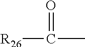

methyl, ethyl or C₁₄-C₂₂ hydrocarbon-based groups,
a hydrogen atom;

R₂₅ is chosen from:
the group

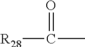

a hydrogen atom;

R₂₄, R₂₆ and R₂₈, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups.

The hydrocarbon-based groups are advantageously linear.

Mention may be made, for example, of the compounds of formula (VI) such as the diacyloxyethyldimethylammonium, diacylo xyethylhydroxyethylmethylammonium, monoacyloxyethyldihydroxyethylmethylammonium, triacyloxyethylmethylammonium and monoacyloxyethylhydroxyethyldimethylammonium salts (chloride or methyl sulfate in particular), and mixtures thereof. The acyl groups preferably contain 14 to 18 carbon atoms and are obtained more particularly from a plant oil, such as palm oil or sunflower oil. When the compound contains several acyl groups, these groups may be identical or different.

These products are obtained, for example, by direct esterification of triethanolamine, triisopropanolamine, an alkyldiethanolamine or an alkyldiisopropanolamine, which are optionally oxyalkylenated, with C₁₀-C₃₀ fatty acids or with mixtures of C₁₀-C₃₀ fatty acids of plant or animal origin, or by transesterification of the methyl esters thereof. This esterification is followed by quaternization using an alkylating agent such as an alkyl (preferably methyl or ethyl) halide, a dialkyl (preferably methyl or ethyl) sulfate, methyl methanesulfonate, methyl para-toluenesulfonate, glycol chlorohydrin or glycerol chlorohydrin.

Such compounds are, for example, sold under the names Dehyquart® by the company Henkel, Stepanquat® by the company Stepan, Noxamium® by the company Ceca or Rewoquat® WE 18 by the company Rewo-Witco.

The composition according to the invention may contain, for example, a mixture of quaternary ammonium monoester, diester and triester salts with a weight majority of diester salts.

It is also possible to use the ammonium salts containing at least one ester functional group that are described in U.S. Pat. No. 4,874,554 and U.S. Pat. No. 4,137,180.

Use may be made of behenoylhydroxypropyltrimethylammonium chloride, provided by Kao under the name Quatarmin BTC 131.

Preferably, the ammonium salts containing at least one ester function contain two ester functions.

Among the quaternary ammonium salts containing at least one ester function, which can be used, it is preferred to use dipalmitoylethylhydroxyethylmethylammonium salts.

Among the above cationic surfactants, it is most particularly preferred to use those of formula (III) and those of formula (VI) and even more preferentially those of formula (III) and in particular cetyltrimethylammonium or behenyltrimethylammonium chlorides.

The above cationic surfactant(s) may be present in an amount ranging from 0.1% to 15% by weight, preferably from 0.5% to 10% by weight and better still from 1% to 5% by weight relative to the total weight of the composition.

The weight ratio according to the invention between the total amount of amphoteric or zwitterionic surfactant(s), on the one hand, and the total amount of cationic surfactant(s), on the other hand, is greater than or equal to 1.2.

Advantageously, the said ratio ranges from 1.2 to 10, preferably from 1.5 to 8 and better still from 2 to 4.

The sulfureous reducing agent(s) present in the composition according to the invention are preferably chosen from organic compounds comprising one or more mercapto groups (—SH), sulfites and sulfite derivatives.

The organic compounds comprising a mercapto group are preferably chosen from thioglycolic acid, thiolactic acid, cysteine, homocysteine, glutathione, thioglycerol, thiomalic acid, 2-mercaptopropionic acid, 3-mercaptopropionic acid, thiodiglycol, 2-mercaptoethanol, dithiothreitol, thioxanthine, thiosalicylic acid, thiodiglycolic acid, lipoic acid, N-acetylcysteine, and thioglycolic or thiolactic acid esters, and mixtures of these compounds.

The term “sulfite derivatives” essentially denotes bisulfites and sulfite diesters of formula R—O—SO₂—R′, with R and R′ denoting C₁-C₁₀ alkyl groups.

The sulfureous reducing agent(s) may be used especially in the form of salts, in particular alkali metal salts such as sodium and potassium salts, alkaline-earth metal salts, for example magnesium and calcium salts, ammonium salts, amine salts and amino alcohol salts.

In a particularly preferred manner, the sulfureous reducing agent(s) are chosen from thioglycolic acid and salts thereof, thiolactic acid and salts thereof, cysteine and salts thereof, alkali metal sulfites, alkali metal bisulfites, and precursors of these sulfites or bisulfites.

Even more preferentially, the sulfureous reducing agent(s) are chosen from thioglycolic acid, cysteine, sodium sulfite, sodium bisulfite and sodium metabisulfite.

The sulfureous reducing agent(s) are preferably present in an amount ranging from 0.1% to 10% by weight, preferably from 0.2% to 5% by weight and in particular from 1% to 5% by weight relative to the total weight of the composition.

The alkaline agent(s) used in the composition according to the invention may be any agent for increasing the pH of the composition in which it is present. The alkaline agent may be a Brönsted-Lowry or Lewis base. It may be mineral or organic.

In particular, the alkaline agent(s) may be chosen from:

a) aqueous ammonia,

b) alkanolamines such as mono-, di- and triethanolamine, isopropanolamine and 2-amino-2-methyl-1-propanol, and also derivatives thereof,

c) oxyethylenated and/or oxypropylenated ethylenediamines,

d) mineral or organic hydroxides,

e) alkali metal silicates such as sodium metasilicates,

f) amino acids, preferably basic amino acids, such as arginine, lysine, ornithine, citrulline and histidine,

g) carbonates and bicarbonates, particularly of a primary amine, secondary amine or tertiary amine, or of an alkali metal or alkaline-earth metal, or of ammonium, and

h) the compounds of formula (VII) below:

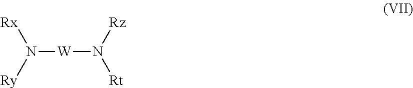

in which W is a C₁-C₆ alkylene residue optionally substituted with a hydroxyl group or a C₁-C₆ alkyl radical; Rx, Ry, Rz and Rt, which may be identical or different, represent a hydrogen atom or a C₁-C₆ alkyl, C₁-C₆ hydroxyalkyl or C₁-C₆ aminoalkyl radical.

Examples of such compounds of formula (VII) that may be mentioned include 1,3-diaminopropane, 1,3-diamino-2-propanol, spermine and spermidine.

The mineral or organic hydroxides are preferably chosen from hydroxides of an alkali metal, hydroxides of an alkaline-earth metal, for instance sodium hydroxide or potassium hydroxide, hydroxides of a transition metal, such as hydroxides of metals from groups III, IV, V and VI of the Periodic Table of the Elements, hydroxides of lanthanides or actinides, quaternary ammonium hydroxides and guanidinium hydroxide.

The hydroxide may be formed in situ, for instance guanidine hydroxide, by reacting calcium hydroxide and guanidine carbonate.

The preferred alkaline agents are in particular aqueous ammonia, ammonium carbonate, ammonium bicarbonate, arginine, monoethanolamine and 2-amino-2-methyl-1-propanol.

The alkaline agent(s) as defined previously may represent, for example, from 0.001% to 20% by weight, and preferably from 0.005% to 10% by weight, relative to the total weight of the composition according to the invention.

The concentration of alkaline agent(s) is especially adjusted as a function of the pH desired for the composition.

Preferably, the composition according to the invention has a pH ranging from 7 to 10 and more preferentially from 7 to 9.5.

The composition according to the invention may also comprise one or more nonionic surfactants and/or one or more anionic surfactants.

Examples of nonionic surfactants that may be used in the composition according to the invention are described, for example, in the Handbook of Surfactants by M. R. Porter, published by Blackie & Son (Glasgow and London), 1991, pp. 116-178. They are especially chosen from alcohols, α-diols and (C₁-C₂₄)alkylphenols, these compounds being polyethoxylated, polypropoxylated and/or polyglycerolated, and containing at least one fatty chain comprising, for example, from 8 to 18 carbon atoms, it being possible for the number of ethylene oxide and/or propylene oxide groups to especially range from 2 to 50, and for the number of glycerol groups to especially range from 2 to 30.

Mention may also be made of copolymers of ethylene oxide and propylene oxide, optionally oxyethylenated sorbitan fatty acid esters, sucrose fatty acid esters, polyoxyalkylenated fatty acid esters, polyoxyalkylenated fatty amides, optionally oxyalkylenated alkyl(poly)glucosides, alkylglucoside esters, derivatives of N-alkylglucamine and of N-acylmethylglucamine, aldobionamides, amine oxides and (poly)oxyalkylenated silicones.

The nonionic surfactants are more particularly chosen from monooxyalkylenated or polyoxyalkylenated and monoglycerolated or polyglycerolated nonionic surfactants, and alkyl(poly)glucosides. The oxyalkylene units are more particularly oxyethylene or oxypropylene units, or a combination thereof, preferably oxyethylene units.

Examples of preferred nonionic surfactants that may be mentioned include:
oxyalkylenated (C₈-C₂₄)alkylphenols;
saturated or unsaturated, linear or branched, oxyalkylenated C₈-C₄₀ alcohols;
saturated or unsaturated, linear or branched, oxyalkylenated C₈-C₃₀ amides;
esters of saturated or unsaturated, linear or branched, C₈-C₃₀ acids and of polyethylene glycols;
saturated or unsaturated, oxyethylenated plant oils;
condensates of ethylene oxide and/or of propylene oxide, alone or as mixtures;
oxyethylenated and/or oxypropylenated silicones;
alkyl(poly)glucosides.

As examples of monoglycerolated or polyglycerolated nonionic surfactants, monoglycerolated or polyglycerolated C₈-C₄₀ alcohols are preferably used.

In particular, the preferred monoglycerolated or polyglycerolated C₈-C₄₀ alcohols correspond to formula (VIII) below:

R₂₉O—[CH₂—CH(CH₂OH)—O]ₘ—H (VIII)

in which formula (VIII):
R₂₉ represents a linear or branched C₈-C₄₀ and preferably C₈-C₃₀ alkyl or alkenyl radical; and
m represents a number ranging from 1 to 30 and preferably from 1 to 10.

As examples of compounds of formula (VIII), mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The alcohol of formula (VIII) may represent a mixture of alcohols in the same way that the value of m represents a statistical value, which means that, in a commercial product, several species of polyglycerolated fatty alcohols may coexist in the form of a mixture.

The alkyl(poly)glycoside nonionic surfactant(s) may be represented by formula (IX) below:

R₃₀O—(R₃₁O)ₜ(G)ᵥ (IX)

in which:

R₃₀ represents a saturated or unsaturated, linear or branched alkyl group comprising from about 8 to 24 carbon atoms, or an alkylphenyl group in which the linear or branched alkyl group comprises from 8 to 24 carbon atoms;

R₃₁ represents an alkylene group containing from about 2 to 4 carbon atoms,

G represents a saccharide unit comprising from 5 to 6 carbon atoms,

t denotes a value ranging from 0 to 10 and preferably from 0 to 4, and

v denotes a value ranging from 1 to 15.

Preferably, the alkyl(poly)glycoside nonionic surfactant(s) correspond to formula (IX) in which:

R₃₀ denotes a linear or branched, saturated or unsaturated alkyl group containing from 8 to 18 carbon atoms,

G denotes glucose, fructose or galactose, preferably glucose,

t denotes a value ranging from 0 to 3, and is preferably equal to 0,

and R₃₁ and v are as defined previously.

The degree of polymerization of the alkyl(poly)glucoside nonionic surfactant(s), as represented, for example, by the index v in formula (IX), ranges on average from 1 to 15 and preferably from 1 to 4. This degree of polymerization more particularly ranges from 1 to 2 and better still from 1.1 to 1.5, on average.

The glycoside bonds between the saccharide units are of 1.6 or 1.4 type and preferably of 1.4 type.

Examples of compounds of formula (IX) that may especially be mentioned are the products sold by the company Cognis under the names Plantaren® (600 CS/U, 1200 and 2000) or Plantacare® (818, 1200 and 2000). Use may also be made of the products sold by the company SEPPIC under the names Triton CG 110 (or Oramix CG 110) and Triton CG 312 (or Oramix® NS 10), the products sold by the company BASF under the name Lutensol GD 70 or the products sold by the company Chem Y under the name AG10 LK.

Use may also be made, for example, of the 1,4-(C₈-C₁₆)alkylpolyglucoside as an aqueous solution at 53% by weight relative to the total weight of the solution, sold by Cognis under the reference Plantacare® 818 UP.

When they are present, the additional nonionic surfactant(s) may represent, for example, from 0.01% to 20% by weight and better still from 0.05% to 10% by weight relative to the total weight of the composition.

The term “anionic surfactant” means a surfactant comprising, as ionic or ionizable groups, only anionic groups. These anionic groups are chosen preferably from the groups CO₂H, CO₂⁻, SO₃H, SO₃⁻, OSO₃H, OSO₃⁻, O₂PO₂H, O₂PO₂H and O₂PO₂²⁻.

The anionic surfactant(s) that may be used in the composition of the invention are chosen especially from alkyl sulfates, alkyl ether sulfates, alkylamido ether sulfates, alkylaryl polyether sulfates, monoglyceride sulfates, alkylsulfonates, alkylamide sulfonates, alkylarylsulfonates, α-olefin sulfonates, paraffin sulfonates, alkylsulfosuccinates, alkyl ether sulfosuccinates, alkylamide sulfosuccinates, alkyl sulfoacetates, acylsarcosinates, acylglutamates, alkylsulfosuccinamates, acylisethionates and N-acyltaurates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group.

Some of these compounds may be oxyethylenated and then preferably comprise from 1 to 50 ethylene oxide units.

The salts of C₆-C₂₄ alkyl monoesters of polyglycoside-polycarboxylic acids may be chosen from C₆-C₂₄ alkyl polyglycoside-citrates, C₆-C₂₄ alkyl polyglycoside-tartrates and C₆-C₂₄ alkyl polyglycoside-sulfo succinates.

When the anionic surfactant(s) are in salt form, they may be chosen especially from alkali metal salts such as the sodium or potassium salt and preferably the sodium salt, ammonium salts, amine salts and in particular amino alcohol salts, or alkaline-earth metal salts such as the magnesium salt.

Examples of amino alcohol salts that may especially be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Use is preferably made of (C₆-C₂₄)alkyl sulfates, (C₆-C₂₄)alkyl ether sulfates, which are optionally ethoxylated, comprising from 2 to 50 ethylene oxide units, and mixtures thereof, in particular in the form of alkali metal salts or alkaline-earth metal salts, ammonium salts or amino alcohol salts. More preferentially, the anionic surfactant(s) are chosen from (C₁₀-C₂₀)alkyl ether sulfates, and in particular sodium lauryl ether sulfate containing 2.2 mol of ethylene oxide.

When they are present, the additional anionic surfactant(s) may represent, for example, from 0.01% to 20% by weight and better still from 0.05% to 10% by weight relative to the total weight of the composition.

Advantageously, the compositions according to the invention may also contain one or more non-silicone fatty substances.

For the purposes of the present invention, the term “fatty substance” means an organic compound which, at room temperature (25° C.) and at atmospheric pressure, is insoluble in water (i.e. it has a solubility in water of less than 1% by weight and preferably less than 0.5% by weight) and is soluble, under the same temperature and pressure conditions, in at least one organic solvent (for example ethanol, chloroform or benzene) to at least 1% by weight.

The term “non-silicone” refers to a fatty substance not comprising in its structure any Si—O—Si sequences.

The fatty substance(s) that may be used in the composition according to the present invention may be chosen especially from plant oils, animal oils, mineral oils, synthetic oils, fatty alcohols, and waxes other than fatty alcohols, and mixtures thereof.

Plant oils that may especially be mentioned include sweet almond oil, avocado oil, castor oil, olive oil, liquid jojoba wax, sunflower oil, wheatgerm oil, sesame oil, groundnut oil, grapeseed oil, soybean oil, rapeseed oil, safflower oil, coconut oil, corn oil, hazelnut oil, palm oil, apricot kernel oil, beauty-leaf oil, evening primrose oil, shea butter, rice bran oil, corn germ oil, passion flower oil and rye oil.

An animal oil that may especially be mentioned is perhydrosqualene.

Liquid paraffin or liquid petroleum jelly may especially be used as mineral oil.

Synthetic oils that may especially be mentioned include squalane, poly(α-olefins), for instance isododecane or isohexadecane, transesterified plant oils, fluoro oils and fatty esters.

The term “fatty esters” denotes compounds of formula RₐCOOR_{b} in which Rₐ represents a linear or branched, hydroxylated or non-hydroxylated, saturated or unsaturated higher acid residue, comprising from 4 to 29 carbon atoms, and R_{b} represents a linear or branched, saturated or unsaturated hydrocarbon-based chain containing from 3 to 30 carbon atoms, the total number of carbon atoms in the ester being greater than 10. Non-limiting examples that may especially be mentioned include Purcellin oil (stearyl octanoate), isopropyl myristate, isopropyl palmitate, butyl stearate, hexyl laurate, isononyl isononanoate, 2-ethylhexyl palmitate, 2-hexyldecyl laurate, 2-octadecyl palmitate, 2-octyldodecyl myristate, isostearyl neopentanoate and tridecyl neopentanoate.

The fluoro oils may be partially hydrocarbon-based and/or silicone-based fluoro oils, for instance those described in document JP-A-2-295 912.

The preferred fatty alcohols comprise, inter alia, myristyl alcohol, cetyl alcohol, stearyl alcohol, arachidyl alcohol, behenyl alcohol and erucyl alcohol.

For the purposes of the present invention, a wax is a lipophilic compound, which is solid at room temperature (about 25° C.), with a reversible solid/liquid change of state, having a melting point greater than about 40° C., which may be up to 200° C., and having in the solid state anisotropic crystal organization. Animal and plant waxes comprise, as essential constituents, long-chain esters of carboxylic acids and of alcohols. In general, the size of the wax crystals is such that the crystals diffract and/or scatter light, giving the composition that comprises them a more or less opaque cloudy appearance. By bringing the wax to its melting point, it is possible to make it miscible with oils and to form a microscopically homogeneous mixture.

As waxes, other than fatty alcohols, that may be used in the composition according to the present invention, mention may be made of waxes of animal origin such as beeswax, spermaceti, lanolin wax and lanolin derivatives; plant waxes such as sunflower wax, rice wax, apple wax, carnauba wax, candelilla wax, ouricury wax, Japan wax, cocoa butter, cork fibre wax or sugar cane wax; mineral waxes, for example paraffin wax, petroleum jelly wax, lignite wax, microcrystalline waxes, ceresin or ozokerite, synthetic waxes such as polyethylene waxes and Fischer-Tropsch waxes, and mixtures thereof.

The non-silicone fatty substance(s) as described above, when they are present in the composition according to the invention, are preferably present in an amount ranging from 0.1% to 30% by weight, preferably from 1% to 20% by weight and better still from 5% to 15% by weight, relative to the total weight of the composition.

Advantageously also, the composition according to the invention may also comprise one or more swelling agents or emollients such as urea, glycols such as butylene glycol, hexylene glycol or propylene glycol, sorbitol, melamine, guanidine or glycerol.

Advantageously also, the composition according to the invention may comprise at least one surfactant, which is preferably nonionic, and/or at least one additional conditioning agent preferably chosen from silicones and/or cationic or amphoteric polymers.

As examples of silicones that may be used as conditioning agent(s) in the compositions of the invention, mention may be made of linear, cyclic, branched or unbranched, volatile or non-volatile silicones. These silicones may be in the form of oils, resins or gums, and may in particular be polyorganosiloxanes.

Organopolysiloxanes are defined in greater detail in Walter Noll's Chemistry and Technology of Silicones (1968), Academic Press. They may be volatile or non-volatile.

When they are volatile, the silicones are more particularly chosen from those with a boiling point of between 60° C. and 260° C.

Conditioning agents that are most particularly preferred include cationic or amphoteric polymers especially such as Polyquaterniums 22, 6, 10, 11, 35 and 37, and hexadimethrine chloride.

The concentration of the additional conditioning agent(s) described above in the composition according to the invention may range from 0.01% to 10% by weight relative to the total weight of this composition, preferably from 0.05% to 5% by weight and even more preferentially from 0.1% to 3% by weight.

The composition in accordance with the invention may also comprise one or more cosmetic adjuvants other than the compounds described previously.

For example, it may comprise one or more standard additives that are well known in the art, such as agents for preventing hair loss, oxidizing agents, vitamins and provitamins including panthenol, sunscreens, mineral or organic pigments, sequestrants, plasticizers, solubilizers, acidifying agents, mineral or organic thickeners, antioxidants, hydroxy acids, nacreous agents, fragrances and preserving agents.

Needless to say, a person skilled in the art will take care to select this or these optional additional compound(s) such that the advantageous properties intrinsically associated with the composition according to the invention are not, or are not substantially, adversely affected by the envisaged addition(s).

The above additives may generally be present in an amount, for each of them, of between 0 and 20% by weight relative to the total weight of the composition.

According to a preferred embodiment, the composition according to the invention is non-colouring, i.e. it does not comprise any direct dyes or oxidation dyes.

A subject of the present invention is also the use of the composition according to the invention.

More particularly, a subject of the present invention is the use of a composition according to the invention for relaxing the curls of keratin fibres and/or for reducing the volume of keratin fibres, preferably of the hair.

In addition, this composition may advantageously be used for cleansing the hair, for straightening the hair or for a combined cleansing and straightening effect.

In particular, the composition according to the invention may be applied before or after a standard hair straightening treatment with or without fixing, so as to facilitate this treatment or reinforce its efficacy (straightening “booster” effect).

It may also be applied after a standard straightening treatment and especially alkaline straightening, so as to maintain the straightening of the hair, which makes it possible, for example, to space out the intervals of the straightening treatments generally performed with more aggressive compositions.

Another subject of the invention is a cosmetic process for treating keratin fibres, which comprises the application to the said wet or dry fibres, one or more times, of a composition as described above.

According to a preferred embodiment, such a process consists in applying to the keratin fibres the composition according to the present invention, and then in optionally rinsing the said fibres after an optional leave-on time.

According to a preferred embodiment, the process according to the invention comprises the following steps:
applying the composition according to the invention to wet hair, and then
leaving the composition to stand on the hair, adhering to a leave-on time ranging from 5 seconds to 60 minutes and preferably from 30 seconds to 15 minutes, at a temperature ranging from 20 to 230° C., preferably from 20 to 60° C. and more preferably from 20 to 30° C., and then
rinsing the hair with water.

The hair may then be dried, for example with a drying hood or a hairdryer, or may be left to dry in the open air.

It is also possible to perform a step of placing the hair under tension, for example by means of a brush, a comb or clips, at the moment when the hair is in contact with the composition according to the invention, or after rinsing the hair.

It is also possible to replace this step of placing under tension with a step of working the head of hair by hand or with a comb.

However, and this constitutes an advantage of the present invention, from the very first application of the composition, including an application without placing the hair under tension, a substantial reduction in the volume of the head of hair is observed. When the hair is curly, relaxation of the curls and/or better curl definition are also observed.

The process of the invention may be performed once.

According to one embodiment, the process according to the invention is repeated several times, until the desired level of straightening is obtained.

Thus, a subject of the present invention is also a process for relaxing hair curls and/or for reducing the volume of the hair, which consists in applying the composition according to the invention one or more times to the hair.

According to a preferred embodiment, the composition is applied several times, either consecutively or after a delay ranging from a few hours to a few days, each application being followed by rinsing, until the desired level of relaxation and/or of volume reduction is obtained.

The application of the composition according to the invention may be followed by an application of one or more rinse-out or leave-in hair compositions, optionally containing a reducing agent or an oxidizing agent.

According to a particular embodiment, the process according to the invention is a process for relaxing the curls of and/or for straightening keratin fibres, such as the hair, which comprises:

a) a step of applying to the keratin fibres the composition as described above; followed by

b) a step of applying to the keratin fibres a second composition (B) comprising one or more oxidizing agents and/or one or more reductones.

The second composition (B) used in this embodiment may comprise one or more oxidizing agents.

The said oxidizing agent(s) are preferably chosen from hydrogen peroxide, urea peroxide, alkali metal bromates or ferricyanides, peroxygenated salts, for instance persulfates, perborates, peracids and precursors thereof and alkali metal or alkaline-earth metal percarbonates, and most particularly hydrogen peroxide.

Preferably, the oxidizing agent(s) are not peroxygenated salts.

The oxidizing agent is preferably hydrogen peroxide.

The oxidizing agent(s) as defined previously may represent, when they are present, from 0.01% to 10% by weight and preferably from 0.1% to 5% by weight, relative to the total weight of composition (B).

The second composition (B) used in this embodiment may comprise one or more reductones.

In a manner known per se, the term “reductone” denotes a compound comprising an enediol structure —(HO)C═C(OH)— adjacent to a carbonyl group >C═O.

Thus, the reductone(s) that may be used in the present invention preferably have the general formula (X):

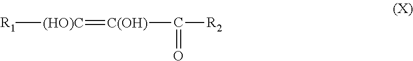

with R₁ and R₂, which may be identical or different, each denoting a group containing at least one carbon and/or oxygen atom, R₁ and R₂ possibly forming with the three carbon atoms of the compound of formula (X) a ring, which is preferably 5- or 6-membered, the additional constituent atoms of which consist of carbon and/or oxygen atoms,

Preferably, R₁ and R₂ form with the three carbon atoms of the compound of formula (X) a ring containing 5 carbon and/or oxygen atoms.

The compound(s) of formula (X) may be in acid form, or in the form of salts, especially in the form of salts of alkali metals such as sodium and potassium, or salts of alkaline-earth metals such as calcium and magnesium, or in the form of esters, especially of C₈ to C₃₀ fatty acids.

In a particularly preferred manner, the compound of formula (X) is a lactone.

The reductone(s) may be chosen especially from reductic acid, ascorbic acid, erythorbic acid and isoascorbic acid, and the salts of these compounds, especially the sodium or potassium salts, ascorbyl palmitate, and mixtures of these compounds.

In a particularly preferred manner, the reductone(s) are chosen from ascorbic acid, erythorbic acid, and the salts of these compounds, especially the sodium or potassium salts.

The reductone(s) may represent, when they are present, from 0.1% to 10% by weight and preferably from 1% to 8% by weight relative to the total weight of composition (B).

When hydrogen peroxide is present in composition (B) used according to the invention, it may also comprise one or more hydrogen peroxide stabilizers.

The second composition (B) used according to the invention may comprise one or more pH regulators, which may be chosen from alkaline agents as described above and/or acidic agents.

The acidic agents that may be used according to the present invention may preferably be chosen from hydrochloric acid, (ortho)phosphoric acid, sulfuric acid, boric acid, and also carboxylic acids, for instance acetic acid, lactic acid or citric acid, or sulfonic acids.

Preferably, composition (B) used according to the invention has a pH ranging from 1 to 8.5, better still from 1 to 5 and more preferentially from 1.5 to 3.

Composition (B) may also contain one or more non-silicone fatty substances.

The non-silicone fatty substance(s) are as described previously for the composition according to the invention.

Composition (B) may also comprise one or more swelling agents or emollients as described previously for the composition according to the invention.

Composition (B) may also comprise at least one additional conditioning agent, preferably chosen from silicones and/or cationic or amphoteric polymers. These compounds are as described previously for the composition according to the invention.

Composition (B) may also comprise one or more cosmetic adjuvants as described previously for the composition according to the invention.

Composition (B) advantageously comprises water or a mixture of water and of one or more cosmetically acceptable solvents as described previously for the composition according to the invention.

According to a preferred embodiment, composition (B) is non-colouring, i.e. it does not comprise any direct dyes or oxidation dyes.

According to a preferred embodiment, the particular process according to the invention comprises the following steps:
applying the composition according to the invention as described previously to wet or dry hair, preferably wet hair, and then
leaving the composition to stand on the hair, adhering to a leave-on time ranging from 1 to 10 minutes and preferably from 2 to 5 minutes, at a temperature ranging from 20 to 230° C., preferably from 20 to 60° C. and more preferably from 20 to 30° C., and then
rinsing the hair with water,
optionally rubbing dry and/or disentangling the hair, and then
applying the second composition (B) as described previously, to the wet hair,
leaving composition (B) to stand on the hair, adhering to a leave-on time ranging from 30 seconds to 10 minutes and preferably from 1 to 3 minutes, at a temperature ranging from 20 to 230° C., preferably from 20 to 60° C. and more preferably from 20 to 30° C., and then
optionally, rinsing the hair with water.

The hair may then be dried, for example by means of a drying hood or a hairdryer, or may be left in the open air.

It is also possible to perform a step of placing the hair under tension, for example by means of a brush, a comb or clips, at the moment when the hair is in contact with the composition according to the invention, or after rinsing the hair.

It is also possible to replace this step of placing under tension with a step of working the head of hair by hand or with a comb.

However, and this constitutes an advantage of the present invention, from the very first implementation of the process according to the invention, including an implementation without placing the hair under tension, a substantial reduction in the volume of the head of hair is observed. When the hair is curly, relaxation of the curls and/or better curl definition are also observed.

It is also found that the unpleasant odours that are given off during the straightening process, or that remain on the straightened hair, are reduced by means of performing the process according to the invention.

The process that is the subject of the present invention, may be performed once.

According to one embodiment, the process according to the invention is repeated several times, until the desired level of straightening is obtained.

According to a preferred embodiment, the process is repeated several times, either consecutively or after a delay ranging from a few hours to a few days.

In addition, this process may advantageously be performed for cleansing the hair, for straightening the hair or for a combined cleansing and straightening effect.

In particular, the process according to the invention may be performed before or after a standard hair straightening treatment with or without fixing, so as to facilitate this treatment or to reinforce its efficacy (straightening “booster” effect).

The process according to the invention may also be performed so as to maintain the straightening of the hair, which makes it possible, for example, to space out the intervals of the straightening treatments generally performed with more aggressive compositions.

A subject of the present invention is also a multi-compartment device or “kit” for relaxing the curls of and/or for straightening keratin fibres, which is suitable for performing the particular process as described above.

The kit according to the invention comprises:
a first compartment containing a composition according to the invention as described above, and
a second compartment containing a composition (B) as described above.

The compositions of this kit are packaged in separate compartments, which may be optionally accompanied by suitable identical or different application means, such as fine brushes, coarse brushes or sponges.

The abovementioned kit may also be equipped with means for dispensing the desired mixture on the hair, such as, for instance the device described in patent FR 2 586 913.

The examples that follow are given purely as illustrations of the present invention.

### EXAMPLES

### Example 1

### Compositions According to the Invention

Three foaming care compositions A, B and C in accordance with the invention were prepared from the ingredients indicated in the table below (in which the contents are indicated in grams of active material):

| Composition | A | B | C |
|---|---|---|---|
| Sodium N-cocoylamidoethyl-N- | 7.5 | 7.5 | 7.5 |
| hydroxyethylaminopropionate⁽¹⁾ | | | |
| Behenyltrimethylammonium chloride⁽²⁾ | 3.16 | 3.16 | 3.16 |
| Pregelatinized hydroxypropyl corn distarch | 3.5 | 3.5 | 3.5 |
| phosphate | | | |
| Cetylstearyl alcohol (50/50 C₁₆/C₁₈) | 7 | 7 | 7 |
| Polydimethylsiloxane containing aminoethyl | 1.1 | 1.1 | 1.1 |
| aminopropyl groups, containing methoxy | | | |
| and/or hydroxyl and alpha-omega silanol | | | |
| functions⁽³⁾ | | | |
| Thioglycolic acid | 2 | — | — |
| Sodium metabisulfite | — | 2 | — |
| Cysteine | — | — | 3 |
| Arginine | — | qs | — |
| | | pH 7 | |
| Monoethanolamine | — | — | qs |
| | | | pH 8 |
| Ammonium bicarbonate | qs | — | — |
| | pH 8 | | |
| Pentasodium pentetate | 0.16 | 0.16 | 0.16 |
| Water | qs 100 | qs 100 | qs 100 |

⁽¹⁾product Rewoteric AM KSF 40 sold by the company Evonik Goldschmidt
⁽²⁾product Varisoft BT 85 sold by the company Evonik Goldschmidt
⁽³⁾product Xiameter MEM-8299 Emulsion sold by the company Dow Corning

The above foaming care compositions were applied to heads of curly and/or voluminous hair, in the following manner: 20 to 30 g of care product were applied to the head of wet hair, i.e. hair that was moistened beforehand and rubbed dry using a terry towel.

The composition was then spread out carefully by massaging the head of hair, so as to work the composition into a lather.

After a leave-on time ranging from 2 to 5 minutes, the hair was rinsed thoroughly with water and then rubbed dry using a towel, disentangled with a comb and dried with a hairdryer.

The Applicant found that the hair was clean, and showed a marked reduction in the volume of the head of hair. As regards curly hair, the Applicant also found that the hair curls were more relaxed, with a better curl definition.

These effects may be obtained after only one application of the composition according to the invention, and without having to straighten the hair with a brush or a clip during drying.

The above treatment process may be repeated several times consecutively on the same head of hair. Enhanced effects are then observed.

### Example 2

### Use of Compositions According to the Invention in a Particular Two-Step Process

The invention was implemented using compositions A to C as described in Example 1 above and using the neutralizing compositions described below.

Two neutralizing compositions in accordance with the embodiment of the invention were prepared from the ingredients indicated in the table below (in which the contents are indicated in grams of active material):

| Composition | D | E |
|---|---|---|
| Diethylenetriaminepentaacetic acid | 0.06 | — |
| Tetrasodium phosphate | 0.02 | — |
| Disodium tin hexahydroxide | 0.04 | — |
| H₂O₂ | 0.6 | — |
| Ascorbic acid | — | 5 |
| pH regulator | qs pH 2.5 | — |
| Fragrance | 0.2 | — |
| Water | qs 100 g | qs 100 g |

Compositions A, B or C above were applied to heads of curly and/or voluminous hair, in the following manner: 30 to 40 g of composition were applied to the head of wet hair, i.e. hair that was moistened beforehand and rubbed dry using a terry towel.

The composition was then spread out carefully by massaging the head of hair, so as to work the composition into a lather.

After a leave-on time ranging from 2 to 5 minutes, the hair was rinsed thoroughly with water and then rubbed dry using a towel and disentangled with a comb.

Composition D or E was then applied to the head of hair, in the following manner: 30 to 40 g of composition were applied to the head of wet hair.

The composition was then spread out carefully by massaging the head of hair.

After a leave-on time of 2 minutes, the hair was rinsed thoroughly with water and then rubbed dry using a towel, disentangled with a comb and dried with a hairdryer.

The Applicant found that the hair showed a marked reduction in the volume of the head of hair. As regards curly hair, the Applicant also found that the hair curls were more relaxed, with a better curl definition.

These effects may be obtained after only one application of the composition according to the invention, and without having to straighten the hair with a brush or a clip during drying.

The Applicant furthermore found that the odour of the hair after treatment was greatly reduced when compared with the prior art treatments.

The above treatment process may be repeated several times consecutively on the same head of hair. Enhanced effects are then observed.

1. Cosmetic composition comprising:
a) one or more amphoteric or zwitterionic surfactants,
b) one or more cationic surfactants,
c) one or more sulfureous reducing agents, and
d) one or more alkaline agents,
the weight ratio between the total amount of the amphoteric or zwitterionic surfactant(s), on the one hand, and the total amount of the cationic surfactant(s), on the other hand, being greater than or equal to 1.2.

2. Composition according to the preceding claim, characterized in that the said amphoteric or zwitterionic surfactant(s) are chosen from derivatives of aliphatic secondary or tertiary amines, optionally quaternized, in which derivatives the aliphatic group is a linear or branched chain comprising from 8 to 22 carbon atoms, the said amine derivatives comprising at least one anionic group, such as a carboxylate, sulfonate, sulfate, phosphate or phosphonate group.

3. Composition according to either of the preceding claims, characterized in that the said amphoteric or zwitterionic surfactant(s) are chosen from (C₈-C₂₀)alkylbetaines, sulfobetaines, (C₈-C₂₀)alkylamido(C₂-C₈)alkylbetaines and (C₈-C₂₀)alkylamido(C₆-C₈)alkylsulfobetaines, and mixtures thereof.

4. Composition according to either of claims 1 and 2, characterized in that the said amphoteric or zwitterionic surfactant(s) are chosen from the compounds having the respective structures (I), (II) and (IIa) below:
Ra-C(O)—NHCH₂CH₂—N⁺(Rb)(Rc)-CH₂COO⁻,M⁺,X⁻ (I)
in which formula (I):
Ra represents a C₁₀-C₃₀ alkyl or alkenyl group derived from an acid Ra-COOH preferably present in hydrolysed coconut oil, or a heptyl, nonyl or undecyl group;
Rb represents a beta-hydroxyethyl group; and
Rc represents a carboxymethyl group;
M⁺ represents a cationic counterion derived from an alkali metal or alkaline-earth metal, such as sodium, an ammonium ion or an ion derived from an organic amine; and
X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylarylsulfonates, in particular methyl sulfate and ethyl sulfate; or alternatively M⁺and X⁻ are absent;
Ra′-C(O)—NHCH₂CH₂—N(B)(B′) (II)
in which formula (II):
B represents the group —CH₂—CH₂—O—X′;
B′ represents the group —(CH₂)_{z}Y′, with z=1 or 2;
X′ represents the group —CH₂—COOH, —CH₂—COOZ′, —CH₂CH₂—COOH or —CH₂CH₂—COOZ′, or a hydrogen atom;
Y′ represents the group —COOH, —COOZ′, —CH₂CH(OH)SO₃H or the group —CH₂CH(OH)SO₃Z′;
Z′ represents a cationic counterion derived from an alkali metal or alkaline-earth metal, such as sodium, an ammonium ion or an ion derived from an organic amine;
Ra′ represents a C₁₀-C₃₀ alkyl or alkenyl group of an acid Ra′-COOH, which is preferably present in coconut oil or in hydrolysed linseed oil, or an alkyl group, especially a C₁₇ group and its iso form, or an unsaturated C₁₇ group;
Ra″-NH—CH(Y″)—(CH₂)ₙ—C(O)—NH—(CH₂)_{n′}-N(Rd)(Re) (IIa)
in which formula (IIa):
Y″ represents the group —COOH, —COOZ″, —CH₂CH(OH)SO₃H or the group —CH₂CH(OH)SO₃Z″;
Rd and Re, independently of each other, represent a C₁-C₄ alkyl or hydroxyalkyl radical;
Z″ represents a cationic counterion derived from an alkali metal or alkaline-earth metal, such as sodium, an ammonium ion or an ion derived from an organic amine;
Ra″ represents a C₁₀-C₃₀ alkyl or alkenyl group of an acid Ra″-COOH, which is preferably present in coconut oil or in hydrolysed linseed oil;
n and n′ denote, independently of each other, an integer ranging from 1 to 3;
and mixtures of these compounds.

5. Composition according to any one of the preceding claims, characterized in that the said amphoteric or zwitterionic surfactant(s) are chosen from cocoylbetaine, cocoylamidopropylbetaine and sodium cocoylamidoethyl-N-hydroxyethylaminopropionate.

6. Composition according to any one of the preceding claims, characterized in that the said amphoteric or zwitterionic surfactant(s) represent from 0.1% to 25% by weight, preferably from 0.5% to 15% by weight and better still from 1% to 10% by weight, relative to the total weight of the composition.

7. Composition according to any one of the preceding claims, characterized in that the said cationic surfactant(s) are chosen from:
those corresponding to the general formula (III) below: 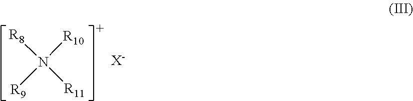
in which R₈ to R₁₁, which may be identical or different, represent a linear or branched, saturated or unsaturated aliphatic group comprising from 1 to 30 carbon atoms, or an aromatic group such as aryl or alkylaryl, at least one of the groups R₈ to R₁₁ denoting a group comprising from 8 to 30 carbon atoms and preferably from 12 to 24 carbon atoms;
X⁻ is an anion chosen from the group of halides, phosphates, acetates, lactates, (C₁-C₄)alkyl sulfates, and (C₁-C₄)alkyl- or (C₁-C₄)alkylarylsulfonates;
quaternary ammonium salts of imidazoline, and, more particularly, those of formula (IV) below: 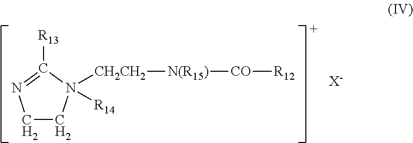
in which R₁₂ represents an alkenyl or alkyl group comprising from 8 to 30 carbon atoms, R₁₃ represents a hydrogen atom, a C₁-C₄ alkyl group or an alkyl or alkenyl group comprising from 8 to 30 carbon atoms, R₁₄ represents a C₁-C₄ alkyl group, R₁₅ represents a hydrogen atom or a C₁-C₄ alkyl group,
X⁻ is an anion chosen from the group of halides, phosphates, acetates, lactates, alkyl sulfates, alkyl- or alkylaryl-sulfonates in which the alkyl and aryl groups preferably comprise, respectively, from 1 to 20 carbon atoms and from 6 to 30 carbon atoms;
quaternary diammonium or triammonium salts, in particular of formula (V): 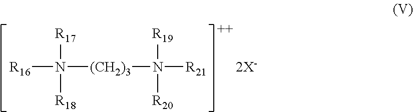
in which R₁₆ denotes an alkyl radical comprising approximately from 16 to 30 carbon atoms, which is optionally hydroxylated and/or interrupted with one or more oxygen atoms, R₁₇ is chosen from hydrogen or an alkyl radical comprising from 1 to 4 carbon atoms or a group (R₁₆ₐ)(R₁₇ₐ)(R₁₈ₐ)N—(CH₂)₃,
R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms; and X⁻ is an anion chosen from the group of halides, acetates, phosphates, nitrates and methyl sulfates;
quaternary ammonium salts containing at least one ester function, such as those of formula (VI) below: 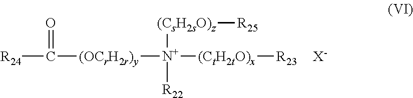
in which:
R₂₂ is chosen from C₁-C₆ alkyl groups and C₁-C₆ hydroxyalkyl or dihydroxyalkyl groups;
R₂₃ is chosen from:
the group 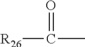
groups R₂₇, which are linear or branched, saturated or unsaturated C₁-C₂₂ hydrocarbon-based groups,
a hydrogen atom,
R₂₅ is chosen from:
the group 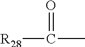
groups R₂₉, which are linear or branched, saturated or unsaturated C₁-C₆ hydrocarbon-based groups,
a hydrogen atom,
R₂₄, R₂₆ and R₂₈, which may be identical or different, are chosen from linear or branched, saturated or unsaturated C₇-C₂₁ hydrocarbon-based groups;
r, s and t, which may be identical or different, are integers ranging from 2 to 6;
y is an integer ranging from 1 to 10;
x and z, which may be identical or different, are integers ranging from 0 to 10;
X⁻ is a simple or complex, organic or mineral anion;
with the proviso that the sum x+y+z is from 1 to 15, that when x is 0 then Rₙ denotes R₂₇, and that when z is 0 then R₂₅ denotes R₂₉.

8. Composition according to any one of the preceding claims, characterized in that the said cationic surfactant(s) represent from 0.1% to 15% by weight, preferably from 0.5% to 10% by weight and better still from 1% to 5% by weight, relative to the total weight of the composition.

9. Composition according to any one of the preceding claims, characterized in that the weight ratio between the total amount of amphoteric or zwitterionic surfactant(s), on the one hand, and the total amount of cationic surfactant(s), on the other hand, ranges from 1.2 to 10, preferably from 1.5 to 8 and better still from 2 to 4.

10. Composition according to any one of the preceding claims, characterized in that the said sulfureous reducing agent(s) are chosen from organic compounds comprising one or more mercapto groups (—SH), sulfites and sulfite derivatives.

11. Composition according to the preceding claim, characterized in that the said sulfureous reducing agent(s) are chosen from thioglycolic acid and salts thereof, thiolactic acid and salts thereof, cysteine and salts thereof, alkali metal sulfites, alkali metal bisulfites, and precursors of these sulfites or bisulfites.

12. Composition according to any one of the preceding claims, characterized in that the said sulfureous reducing agent(s) represent from 0.1% to 10% by weight and preferably from 0.2% to 5% by weight relative to the total weight of the composition.

13. Composition according to any one of the preceding claims, characterized in that the said alkaline agent(s) are chosen from:
a) aqueous ammonia,
b) alkanolamines and derivatives thereof,
c) oxyethylenated and/or oxypropylenated ethylenediamines,
d) mineral or organic hydroxides,
e) alkali metal silicates,
f) amino acids, which are preferably basic amino acids,
g) carbonates and bicarbonates, particularly of a primary amine, secondary amine or tertiary amine, or of an alkali metal or alkaline-earth metal, or of ammonium, and
h) the compounds of formula (VII) below: 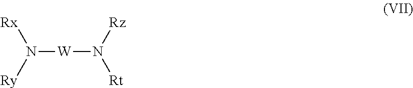
in which W is a C₁-C₆ alkylene residue optionally substituted with a hydroxyl group or a C₁-C₆ alkyl radical; Rx, Ry, Rz and Rt, which may be identical or different, represent a hydrogen atom or a C₁-C₆ alkyl, C₁-C₆ hydroxyalkyl or C₁-C₆ aminoalkyl radical.

14. Composition according to any one of the preceding claims, characterized in that the pH ranges from 7 to 10 and more preferentially from 7 to 9.5.

15. Composition according to any one of the preceding claims, characterized in that it comprises at least one surfactant, which is preferably nonionic, and/or at least one additional conditioning agent preferably chosen from silicones and/or cationic or amphoteric polymers.

16. Use of a composition as defined in any one of the preceding claims, for relaxing the curls of keratin fibres and/or for reducing the volume of keratin fibres, preferably of the hair.

17. Cosmetic process for treating keratin fibres, comprising the application to the said wet or dry fibres, one or more times, of a composition as defined in claims 1 to 15.

18. Cosmetic process according to the preceding claim, comprising the following steps:
applying the composition according to claims 1 to 15 to wet hair, and then
leaving the composition to stand on the hair, adhering to a leave-on time ranging from 5 seconds to 60 minutes and preferably from 30 seconds to 15 minutes, at a temperature ranging from 20 to 230° C., preferably from 20 to 60° C. and more preferably from 20 to 30° C., and then
rinsing the hair with water.

19. Process for relaxing the curls of and/or for straightening keratin fibres, such as the hair, characterized in that it comprises:
a) a step of applying to the keratin fibres the composition as defined in any one of claims 1 to 15; followed by
b) a step of applying to the keratin fibres a second composition (B) comprising one or more oxidizing agents and/or one or more reductones.

20. Process according to claim 19, characterized in that composition (B) comprises one or more oxidizing agents preferably chosen from hydrogen peroxide, urea peroxide, alkali metal bromates or ferricyanides and peroxygenated salts, and, more preferentially, the oxidizing agent is hydrogen peroxide.

21. Process according to claim 20, characterized in that the said oxidizing agent(s) represent from 0.01% to 10% by weight and preferably from 0.1% to 5% by weight, relative to the total weight of composition (B).

22. Process according to any one of claims 19 to 21, characterized in that composition (B) comprises one or more reductones, preferably chosen from the compounds of general formula (X): 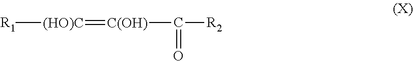
with R₁ and R₂, which may be identical or different, each denoting a group containing at least one carbon and/or oxygen atom, R₁ and R₂ possibly forming with the three carbon atoms of the compound of formula (X) a ring, which is preferably 5- or 6-membered, the additional constituent atoms of which consist of carbon and/or oxygen atoms,
the compound(s) of formula (X) being in acid form, or in the form of salts, especially in the form of salts of alkali metals or of alkaline-earth metals, or in the form of esters, especially of C₈ to C₃₀ fatty acids.

23. Process according to claim 22, characterized in that the said reductone(s) are chosen from reductic acid, ascorbic acid, erythorbic acid and isoascorbic acid, and the salts of these compounds, especially the sodium or potassium salts, ascorbyl palmitate, and mixtures of these compounds.

24. Process according to either of claims 22 and 23, characterized in that the said reductone(s) represent from 0.1% to 10% by weight and preferably from 1% to 8% by weight relative to the total weight of composition (B).

25. Multi-compartment device or kit for relaxing the curls of and/or for straightening keratin fibres, comprising at least two compartments:
a first compartment containing a composition as defined in any one of claims 1 to 15, and
a second compartment containing a composition (B) as defined in any one of claims 19 to 24.
